(19) United States
(12) Patent Application Publication
(10) Pub. No.: US 2013/0251697 A1
(43) Pub. Date: Sep. 26, 2013

(54) NOVEL NASAL SPRAY

(71) Applicant: Ford D. Albritton, IV, Dallas, TX (US)

(72) Inventor: Ford D. Albritton, IV, Dallas, TX (US)

(21) Appl. No.: 13/706,085

(22) Filed: Dec. 5, 2012

Related U.S. Application Data
(63) Continuation of application No. 12/484,976, filed on Jun. 15, 2009, now abandoned.

(51) Int. Cl. A61K 36/82; A61K 31/05; A61K 31/047; A61K 31/352; A61K 31/198; A61K 31/385; A61K 31/375

(52) U.S. Cl. CPC A61K 36/82; A61K 31/385; A61K 31/05; A61K 31/375; A61K 31/352; A61K 31/198; A61K 31/047
USPC 424/94.1; 514/440; 424/729

(57) ABSTRACT

Systems and methods are disclosed for treating a plurality of sinsousal conditions to be treated. These systems and methods include selecting a combination of at least two compounds that are capable of addressing a plurality of sinsousal conditions. At least one of the at least two compounds is selected from a group of anti fungals, antioxidants, and chelators. These systems and methods also include verifying that the compounds are biologically compatible and creating a combination compound using the selected compounds.

### CROSS-REFERENCE TO RELATED APPLICATION(S) AND CLAIM OF PRIORITY

The present application is related to U.S. Provisional Patent No. 61/131,959, filed Jun. 13, 2008, entitled “NOVEL NASAL SPRAY”. Provisional Patent No. 61/131,959 is assigned to the assignee of the present application and is hereby incorporated by reference into the present application as if fully set forth herein. The present application hereby claims priority under 35 U.S.C. §119(e) to U.S. Provisional Patent No. 61/131,959.

### TECHNICAL FIELD

The present invention relates to a multi bio-systematic approach to a plurality of areas of sinonasal health.

### BACKGROUND

Over the past few decades, free radicals, highly reactive and thereby destructive molecules, are known increasingly for their importance to human health and disease. Many common and life threatening human diseases, including atherosclerosis, diabetes, cancer, and aging, have free radical reactions as an underlying mechanism of injury. Because the human body is continuously exposed to free radicals and other Reactive oxygen species (ROS), from both external sources (sunlight, other forms of radiation, pollution) and endogenously generated sources, ROS-mediated tissue injury is a final common pathway for a number of disease processes. Radicals of oxygen (superoxide anion, hydroxyl radical, and peroxy-radicals), reactive non-radical oxygen species such as hydrogen peroxide and singlet oxygen, as well as carbon, nitrogen, and sulfur radicals comprise the variety of reactive molecules that can cause damage to cell.

The conceptual understanding of the interaction of such reactive oxygen species (ROS) with living organisms has undergone a remarkable evolution. Antioxidants are intimately involved in the prevention of cellular damage—the common pathway for cancer, aging, and a variety of diseases. These molecules safely interact with free radicals and terminate the chain reaction before vital molecules are damaged. They neutralize free radicals by donating one of their own electrons. The antioxidant nutrients themselves do not become free radicals by donating an electron because they are stable in either form; they act as scavengers, helping to prevent cell and tissue damage that could lead to cellular damage and disease. Although there are several enzyme systems within the body that scavenge free radicals, the principle antioxidants are vitamin E, vitamin C, n-acetylcysteine and -lipoic acid. The vitamins C and E are thought to protect the body against the destructive effects of free radicals. Additionally, selenium, a trace metal that is required for proper function of one of the body's antioxidant enzyme systems, is also included in this category.

### SUMMARY OF THE INVENTION

A method is disclosed for treating a plurality of sinsousal conditions. This method includes selecting a combination of at least two compounds that are capable of addressing a plurality of sinsousal conditions. At least one of the two compounds is selected from a group of anti fungals, antioxidants, and chelators. This method also include verifying that the compounds are biologically compatible and creating a combination compound using the selected compounds.

Before undertaking the DETAILED DESCRIPTION OF THE INVENTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 represents an overview of one method of identifying and treating a plurality of conditions according to an embodiment of this disclosure.

FIG. 2 illustrates a flowchart for creating a compound for treating a plurality of conditions according to an embodiment of this disclosure.

It may be advantageous to set forth definitions of certain words and phrases that may be used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

### DETAILED DESCRIPTION OF THE INVENTION

Chronic sinonasal disease has micro and macroscopic etiologies often working in concert. Surgical advances have allowed better control of the macro features, but not of the micro features. Mucosal diseases are marked by an incomplete understanding of the reasons behind them and the gaps in medical therapy currently in place. There are several causes of mucosal and submucosal inflammatory problems including, but not limited to cytotoxic substances from eosinophils, lymphocytes, superoxides and other ROS, cytokine release, alterations in Nitric Oxide Synthase (NOS) function, bacterial infection, inflammation from allergy, and autoimmune disease.

In order to address these problems, one innovative approach is to fill gaps left between classic therapies of topical steroids and antihistamines with compounds that work at the subcellular level. Several naturally occurring molecules classified as “nutraceuticals” have existed in homeopathy and dietary heath for millennia and are poorly used or trusted in allopathy. Biochemical research has elucidated the mechanisms at play for many of these compounds. One of the innovative elements of the present disclosure is to use these compounds to address sinonasal problems. Several of these compounds have a promising potential to fill some of the current gaps in successful therapy. Some of the compounds that can address sinonasal problems, based upon research conducted in conjunction with the present disclosure, include resveratrol, Green Tea, quercetin, xylitol, L-fucose, and D-galactose.

In order to address the sinonasal problems discussed above, compounds need to be created that protect the cellular integrity and physiologic function through mechanisms on a tissue, cellular, and sub cellular level. These mechanisms need to treat a plurality of conditions, with each condition being treated by at least one approach and at least one mechanism. The use of a plurality of mechanisms simultaneously addresses chronic sinonasal problems in a uniquely effective way. These mechanisms include, but are not limited to, mechanisms that decrease proinflammatory leukotrienes and prostaglandins, and decrease matrix metalloproteinase 9 [(MMP-9)-actor in tissue injury and death and microvascular injury]. In addition, these mechanisms provide for cytoprotection from hazardous metabolites including, but not restricted to, oxygen free radicals, immunologic modification to prevent immune mediated damage to inner ear cells, promoting efficient and appropriate cell messenger system operation (for example, NO (nitric oxide), Ceramide synthases). Other mechanisms include those that provide for the regulation and repair of mucociliary transit and promote health of the nasal cilia allows decreased mucus stagnation. The decrease in stagnant mucus reduces microbe colonization and secondary immune response to these microbes.

In addition mechanisms that provide for the anti-microbial function (xylitol, fucose, galactose) reducing microbial colonization in the nose also reduce the mucosal inflammatory potential.

These mechanisms promote cell membrane stability and assist in the regulation of apoptosis (planned cell death at senescence). These mechanisms can further provide for the protection and facilitation of mitochondrial function, health, and stability. These mechanisms may promote efficient electron transfer, protect from age related dysfunctional ETC by electron acceptance, modulate enzymes to improve the protective benefit of the enzymes, and modulate platelet functions which may include (but not restricted to) reduction of thrombosis of macro and microcirculation.

The disclosed compounds are useful in the promotion of numerous biologic/medical situations. Through the disclosed compounds, a multi bio-systematic approach to most, if not all, areas of sinonasal health has been discovered. The above mechanisms and approaches to alleviating sinuous problems listed should be addressed as a collective effort to approach a plurality of problems simultaneously. It is understood that the prevention of an individual problem may not be sufficient to alleviate sinsousal problems.

It is understood that a synergistic relation between all of the ingredients in concert that optimize normal mucosal physiology, reduce aberrant immunologic over activity, and reduce cascade of events leading to inflammation.

For the purpose of clarity, two terms will be briefly defined herein. “Primary compound” is intended to refer to a compound that is comprised of a plurality of other compounds (e.g. one or more sub-compounds, or a mixture of heterogeneous compounds). “Sub-compounds” are intended to refer to the one or more compounds that make up the primary compound.

One example of a primary compound might be a compound comprised of the following sub-compounds:

### Anti Fungals/Virals:

### Monolauren

### Antioxident/Immunomodulators:

### Resveratrol

### Quercetin

Acetyl carnitine
Green tea extract

### Ubiquinol

### NAC

### Chelators:

### Zinc

It is understood that any concentration of any of the compounds listed above, described herein, or operating substantially with either the compounds listed above or described herein may be used consistent with this disclosure.

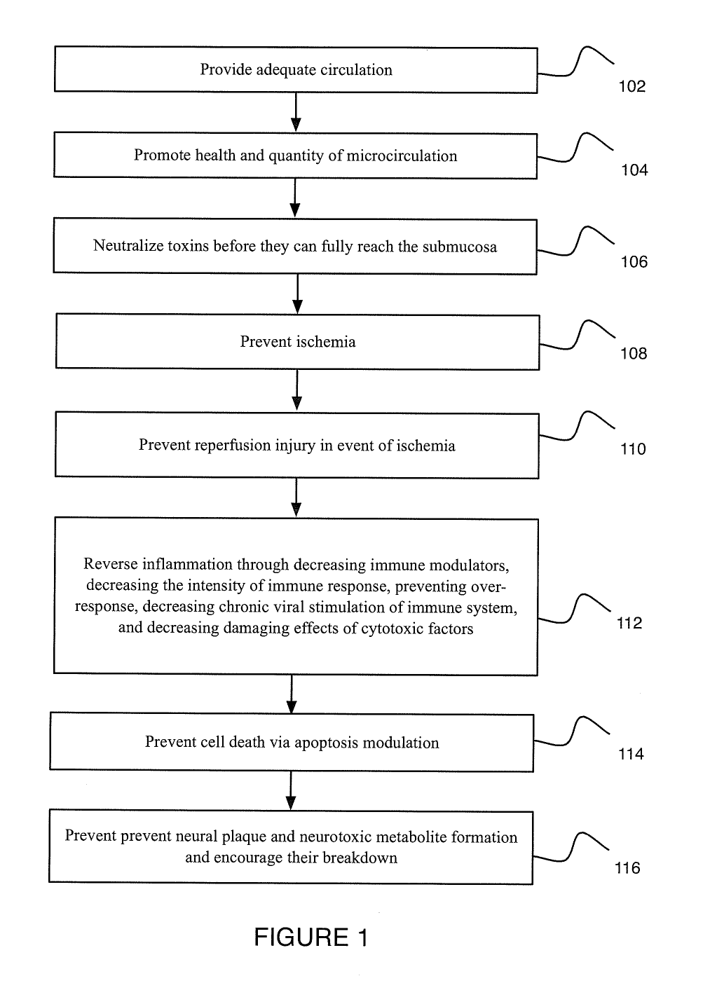

Therefore, there is a unique and novel approach to the reduction of sinsousal problems. In one embodiment, this approach may be undertaken as described below in terms of a plan that has a number of ordered steps. While the potential benefits of simultaneous approaches to assist a plurality of mechanisms has been mentioned above, it is understood that in some embodiments a step-wise mechanism with a planned order of steps may be used in order to alleviate sinsousal problems. The steps listed below may be performed in any order, including the order illustrated by the flowchart 100 shown in FIG. 1. In block 102, adequate circulation is provided to the nasal area. In block 104, the health and quantity of microcirculation is promoted. In block 106, the toxins are neutralized before they can fully reach the submucosa (e.g., by metal chelation). In block 108, ischemia is prevented. In block 110, in the event of ischemia, injury from reperfusion is prevented through antioxident activity and native ROS scavengers. The use of resources during period of cell stress may be reduced during this block, or any other block.

In block 112, inflammation is reversed by decreasing immune modulators, decreasing the intensity of immune response, and preventing over-response. In addition, inflammation is further reduced by decreasing chronic viral stimulation of immune system, and decreasing damaging effects of cytotoxic factors. In block 114, cell death via apoptosis modulation is prevented. In block 116, neural plaque and neurotoxic metabolite formation is prevented and their breakdown is encouraged (important in sense of smell maintenance).

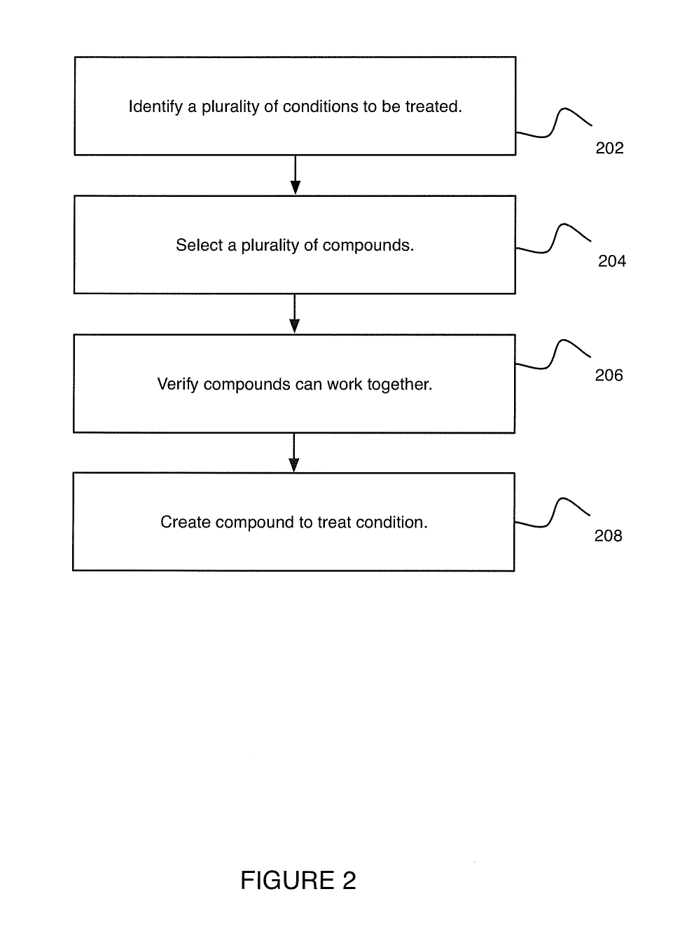

FIG. 2 is an example of one method of selecting the subcompounds to be used in treating a plurality of sinonasal health. In block 202, a plurality of conditions to be treated is identified. In block 204, a plurality of compounds is selected to treat the plurality of conditions. In block 206, the combination of compounds is verified to be safe for use in a medicinal application. For the purpose of clarity, the phrase “biologically compatible” will be used to refer to a combination of compounds that is safe for use together. In block 208, the compound is created.

All factors aim to prevent problems from a micro to macro level: cellular, organ, and neural integrated system. Described below are compounds and sub-compounds that are directed towards the protection of cells and the prevention of deterioration. It is expressly understood that this combination or combinations of compounds may be used individually or together to alleviate sinsousal problems.

One example of a compound that is contemplated as being used as a sub-compound is Alpha Lipoic Acid (ALA). It is understood that ALA is a potent antioxidant that may decrease inflammatory cascade and reduce tissue remodeling disturbance which may lead to polypogenesis. In addition, ALA may be an inhibitor of matrix metalloproteinase-9 (MMP)-9. MMP's may lead to disturbance in submucosal tissue and have been implicated in polyp formation.

MMP's are also associated in the breakdown of the “extracellular matrix” which might trigger polyposis.

Another example of a compound that is contemplated as being used as a sub-compound is Acetyl-L-Carnitine. It is understood that Acetyl-L-Carnitine may be an inducer of cytoprotective protein/enzymes (with native antioxidant effects) such as hemeoxygenase-1, Hsp 70, Super Oxide Dismutase-2, Glutathione synthase (GSH). In addition, Acetyl-L-Carnitine may be an inducer of metabolism of acetyl that may result in reducing cellular ischemia time and the increase available Adenosine triphosphate (ATP). Acetyl-L-Carnitine may also inhibit neuronal “exitotoxicity” which is effective in reducing neural hyper repolarization. Cells require less oxygen/glucose during a state of deficiency/absence and provides for membrane stabilization. In addition, Acetyl-L-Carnitine may be effective in the processes of metal chelation, (e.g., the removal of metals). This results in the reduction of cell death/injury from hypoxia and other sources, as well as reduces damage to mucosa and submucosa by reducing the exposure of healthy tissue to immune derived superoxides and other chemicals released in inflammatory cascade targeting pathogens but giving collateral damage to surrounding tissues. It is understood that Acetyl-L-Carnitine may also be used to prevent mitochondrial aging and decrease oxidative damage by decreasing protein carbonyls (protein carbonyls are markers of protein injury and oxidative stress) and decreasing hydroxynonenal (HNE) marker of lipid peroxidation.

Yet another example of a compound that is contemplated as being used as a sub-compound is Resveratrol. Resveratrol may be used as an anti inflammatory, and may decrease the following proinflammatory mediators:

Tumor Necrosis Factor alpha (TNF-a)

Prostaglandin PG E2

COX2 (cyclooxygenase pathway inflammatory mediator . . . target of COX2 inhibitors such as Bextra/celebrex)

NF Kappa-B signaling pathway

Interleukins −1,6

Also decreases overall neutrophil migration (white blood cell involved in infection/inflammation).

In addition, Resveratrol may inhibit mast cell modulator release at concentrations, (for example, within a range including, but not limited to, 10-100 mMol, and Resveratrol may decrease the following:

PG D2

Histamine

TNF-a

In addition, Resveratrol scanvenges ROS via NOS (nitric oxide synthase) which increases NO, and has many advantages including NO scavenges OH— and superoxides, NO is more powerful than superoxide dismutase (SOD), and NO+SOD work synergistically to clear reactive oxygen species (ROS). In addition, Resveratrol also Scanvenges ROS via AhR (aryl hydrocarbon receptor) which results in Resveratrol binding as a competitive antagonist cause preventing nuclear translocation which provides an antiproliferative effect.

In addition, Resveratrol may be used as an anti viral (anti neuro trophic virals) which provides for the reversible inhibition Herpes simplex virus (HSV) 1 & 2 replication and decreases intercellular adhesion molecule (ICAM) selectin. This allows for the adhesion of molecules causing inflammation and leukocyte migration that may be mediated through a NO pathway and decrease Interleukin-1 (IL-1), tumor neurosis factor alpha (TNF-a), via direct inhibition of their transcription. This direct inhibition may be performed through arachnidonic acid compounds such as Thromboxane B2 (TxB2), hydroxyheptadecatrienoic acid (HHT), and 12 L-Hydroxyeicosatetraenoic (12-HETE).

In addition, Resveratrol may be used in the anti apoptosis in cardiomyocytes, and may perform a potentially similar action in olfactory nerves (sense of smell). This may allow for the use of the adenosine A1 receptor agonist that may decrease glucose use in the olfactory nucleus of brain and olfactory bulb. This is a novel and unique approach to decreasing glucose use in the olfactory nucleus of the brain and olfactory bulb. Resveratrol may also be used in the olfactory nerve for protein (PKC, tyrosine kinases as examples) and mitochondrial ATP-sensitive K+ channel.

In addition, Resveratrol decreases B-Amyloid and encourages its breakdown as well as increases genes SIRT1 expression [up to 13 fold] (life extending genes) and increase cellular life span (33%-50% in animal studies dose dependent).

Yet another example of a compound that is contemplated as being used as a sub-compound is L-Cysteine. It is understood that L-Cysteine protects olfactory cells by increasing the intracellular glutathione (GSH) levels via decreasing the effect of glutamate. Glutamate-cysteine ligase (GCL), previously known as gamma-glutamylcysteine synthetase, is the rate-limiting enzyme for GSH synthesis. An elevated Glu/Cys ratio causes damaging NO increases, which in turn causes chemical exposure and induces GCL expression.

Yet another example of a compound that is contemplated as being used as a sub-compound is Epigallocatechin gallate (EGCG) [10-100 mM]. EGCG is an antioxidant that reacts with ROS and activates natural pathways. EGCG also acts as a neuroprotectant that allows for the protection in spiral ganglion cells and upregulates Mn-superoxide dismutase (MnSOD) as well as protects against H2O2. ECGC also reduces metal toxicity chelation.

One of the novel features of the present primary compound is that when compounds such as EGCG are coupled with Resveratrol, there is a decrease in leukemic cell line proliferation which may be helpful in allergy, mast cell and, eosinophil control. This may decrease NF-Kappa B and cleaved capsase-3 (end stage protease in apoptosis).

EGCG may also be used to reduce IFN (interferon) through enhancing viral protection activity. This allows for the immune modulating with the IFN stimulate the “JAK/STAT1” pathway.

EGCG may also be used as an anti-inflammatory that induces cell cycle arrest in certain T cells, blocks enzymes making t cell growth factors and effectors, and decreases TNF-A production (Tumor Necrotic Factor). The ECGC may then inhibit I kappa B kinase (IKK) and lead to decreased nuclear factor-kappa B (NF-kappa B) and TNF-A. In addition, EGCG may inhibit IL-1B and its signal transduction by decreasing IL-8 (major neutrophil attractant) and decreasing NF-kappa B.

One of the novel features of the present disclosure is in the presently disclosed combination of the ECGC and the Resveratrol. This combination allows several steps in the inflammatory cascade to be modulated to effectively reduce inflammatory messaging in a non-additive way as well as inhibit certain types of polyp growth.

Yet another example of a compound that is contemplated as being used as a sub-compound is Catechin. It is understood that Catechin has antioxidant ability, maintains glutathione, and has anti-inflammatory properties. These anti-inflammatory properties are evident through the blocking of IL-1, IL-6, inhibition of arachnidonic acid metabolism through both cyclooxygenase (COX) and lipoxygenase pathways.

Yet another example of a compound that is contemplated as being used as a sub-compound is Xylitol. It is understood that Xylitol has native antimicrobial function in nasal mucosa and decreases osmolarity of nasal secretions (both by decreasing nasal secretion viscosity and improving ciliary flow in patients with defects of nasal mucosal chloride pumps by increasing water content in the mucous of these patients and keeping it from stagnating).

Yet another example of a compound that is contemplated as being used as a sub-compound is fucose (or galactose). It is understood that fucose, when combined with galactose or alone, has the ability to restore ciliary function through reversing paralysis caused by bacterial by products and promoting proper function

Yet another example of a compound that is contemplated as being used as a sub-compound is Coenzyme Q10. It is understood that Coenzyme Q10 may be used as an antioxidant against superoxides and prevents lipid peroxidation as well as scavenges —OH radicals. Coenzyme Q10 also inhibits free radical formation by aged mitochondrial enzymes ar-ECTO-NOX) [ar=age related] by direct enzyme activity blockage not dependent on ROS scavenging. Coenzyme Q10 prevents CoA reductase inhibitor induced neurologic side effects, increases brain mitochondrial concentration, and prevents mitochondrial dysfunction

Yet another example of a compound that is contemplated as being used as a sub-compound is Resveratrol combined with citric flavanoids. It is understood that flavanoids enhance bioavailability of Resveratrol through inhibition of sulfation. Resveratrol is sulfated, and the hepatic and duodenal sulfation might limit the bioavailability of this compound. The addition of flavanoids has a primary biologic benefit alone but with Resveratrol, it also increases bioavailability of the Resveratrol.

It is also understood that the combination of Resveratrol and catechins (green tea) creates a synergistic effect against beta-Amyloid Protein (1-41) toxicity (plaque in Alzheimer's and other neurodegenerative diseases). Results demonstrate that Resveratrol and catechin have different activities on the signal transduction pathway involving protein phosphorylation. These effects include:

synergistic effect against beta-Amyloid Protein (1-41) toxicity;
different activity of resveratrol and catechin on signal transduction pathways;
metal chelation;
partition coefficient between water and lipids;
hydrogen donation redox potential; and
enzyme inhibition.

It is explicitly understood that cooperation of the ECGC and the Resveratrol allows several steps in the inflammatory cascade to be modulated at several layers of the cascade.

In some embodiments, coenzyme Q10 with agents down regulating NF-Kappa can provide synergistic neuroprotection. Examples of NF-Kappa depressors include EGCG, Resveratrol, and metals: such as selenium, zinc, and Mn. In other embodiments, atechins with cysteine generate neuroprotective antioxidant compounds and protect against glutamate induced cell death during cell stress and enhance glutathione activity.

In yet another embodiments, IV MMP inhibitors may be used with ALA, Resveratrol, & green tea. These IV MMP inhibitors inhibit production of proinflammatory MMP's which contribute to polyp growth and nasal inflammation.

In one embodiment of the present disclosure, a compound is contemplated with the following approximate percentages of active ingredients:

| | |
|---|---|
| Quercentin Calcone Powder | .3% |
| Resveratrol 50% powder | .15% |
| EDGC_Green Tea 90% Powder | .3% |
| Lipoic Acid-R Powder | .3% |
| Ascorbic Acid USP powder | .3% |
| Glutathione L. Reduced | .4% |
| Acetyl-C-Cysteine | .3% |
| Xylitol | 2% |

This example is intended for the purpose of illustrating one combination of compounds useful in treating sinsoual conditions. It is expressly understood that any combination may be used consistent with the present disclosure.

Although the present invention and its advantages have been described in the foregoing detailed description and illustrated in the accompanying drawings, it will be understood by those skilled in the art that the invention is not limited to the embodiment(s) disclosed but is capable of numerous rearrangements, substitutions and modifications without departing from the spirit and scope of the invention as defined by the appended claims.

1.-13. (canceled)

14. A chemical composition, comprising:
a combination of at least two compounds, capable of addressing a plurality of sinonasal conditions, wherein at least one of the at least two compounds is selected from a group of anti fungals, antioxidants, and chelators, and wherein the at least two compounds are biologically compatible; and wherein the composition comprises quercetin chalcone and resveratrol.

15. The composition in accordance with claim 14, wherein the composition comprises epigallocatechin gallate (ECGC).

16. The composition in accordance with claim 14 wherein the composition further comprises: monolauren, epigallocatechin gallate (ECGC), lipoic acid, acetyl-c-cysteine and zinc.

17. A chemical composition, comprising:
a combination of biologically compatible compounds operable for addressing a plurality of sinonasal conditions, the combination of compounds comprising:
an anti fungal;
at least one antioxidant; and
a chelator.

18. The composition in accordance with claim 17 wherein the combination of compounds comprises: resveratrol, quercetin, acetyl carnitine and green tea extract.

19. The composition in accordance with claim 18 wherein the resveratrol, quercetin, acetyl carnitine and green tea extract together comprise approximately between 0.2% and 5% of the combination of compounds.

20. The composition in accordance with claim 17 wherein the antifungal comprises monolauren.

21. The composition in accordance with claim 20 wherein the chelator comprises zinc.

22. The composition in accordance with claim 21 wherein the combination of compounds further comprises lipoic acid.

23. The composition in accordance with claim 18 wherein the quercetin is in the form of quercetin chalcone powder in an amount of approximately 0.3% of the combination of compounds.

24. The composition in accordance with claim 23 wherein the resveratrol is in an amount of approximately 0.15% of the combination of compounds.

25. The composition in accordance with claim 24 wherein the combination of compounds further comprises approximately 2% xylitol.

26. A chemical composition, comprising:
a combination of biologically compatible compounds operable for addressing a plurality of sinonasal conditions, the combination of compounds comprising:
an anti fungal;
a group of antioxidants, the group of antioxidants comprising at least three antioxidants; and
a chelator.

27. The composition in accordance with claim 26 wherein the group of antioxidants comprises quercetin, resveratrol, and epigallocatechin gallate (ECGC), and the combination of compounds further comprises: lipoic acid, ascorbic acid, glutathione, acetyl-c-cysteine and xylitol.

28. The composition in accordance with claim 27 wherein the anti fungal comprises monolauren and the chelator comprises zinc.

29. The composition in accordance with claim 26 wherein the combination of compounds comprises;
quercetin in an amount of approximately 0.3% of the combination of compounds;
resveratrol is in an amount of approximately 0.15% of the combination of compounds;
lipoic acid in an amount of approximately 0.3% of the combination of compounds;
glutathione in an amount of approximately 0.3% of the combination of compounds;
acetyl-c-cysteine in an amount of approximately 0.3% of the combination of compounds; and
zylitol in an amount of approximately 2% of the combination of compounds.
